(19) United States
(12) Patent Application Publication
(10) Pub. No.: US 2018/0361126 A1
(43) Pub. Date: Dec. 20, 2018

(54) SYSTEMS AND METHODS FOR BODY LUMEN DRAINAGE

(71) Applicant: Boston Scientific Scimed, Inc., Maple Grove, MN (US)

(72) Inventor: Martyn G MR Folan, Galway (IE)

(21) Appl. No.: 16/011,825

(22) Filed: Jun. 19, 2018

Related U.S. Application Data
(60) Provisional application No. 62/522,342, filed on Jun. 20, 2017.

(51) Int. Cl. A61M 27/00

(52) U.S. Cl. CPC A61M 27/002; A61M 2205/0266; A61M 2210/106; A61M 2210/1053; A61M 2210/1075; A61M 2210/1057; A61M 2210/1082

(57) ABSTRACT

The present disclosure relates generally to the field of medical devices and establishing fluid communication between body lumens. In particular, the present disclosure relates to minimally invasive devices and methods for body lumen access and/or drainage, and devices and methods for creating an open flow passage between two or more body lumens.

### CROSS REFERENCE TO RELATED APPLICATIONS

The present application claims the benefit of priority under 35 U.S.C. § 119 to U.S. Provisional Application Ser. No. 62/522,342, filed on Jun. 20, 2017, the disclosure of which is incorporated by reference in its entirety for all purposes.

### FIELD

The present disclosure relates generally to the field of medical devices and establishing fluid communication between body lumens. In particular, the present disclosure relates to devices and methods for establishing drainage between two or more body lumens and/or within a body vessel.

### BACKGROUND

Conventional plastic stents are available in a variety of configurations designed to establish or maintain flow between body lumens and/or within body vessels. As compared to metallic stents, the low cost, pushability through tortuous anatomies and ability to be repositioned for accurate placement, makes plastic stents a preferred option for many body lumen or vessel drainage procedures. To address the inherent tendency of plastic stents to become occluded, physicians often place multiple plastic stents between the body lumens (or within the body vessel) to be drained. Depending on the specific medical scenario, a physician may place as many as 10 stents within a patient during a single procedure. In addition to providing extra lumens for intrinsic drainage through each stent, the presence of multiple stents provides extrinsic flow (e.g., ancillary capillary flow) along and/or between the outer surfaces of each stent.

A variety of advantageous medical outcomes may therefore be realized by the devices and/or methods of the present disclosure, which provide the combined benefits of intrinsic and extrinsic body lumen or vessel drainage in a single medical device.

### SUMMARY

In one aspect, the present disclosure relates to a medical device comprising an elongate tubular body which includes a proximal end and a distal end, with a first elongate portion of the elongate tubular body comprising an outer surface and an inner surface defining a first channel along a length of the elongate tubular body, and a second elongate portion of the elongate tubular body comprising an outer surface and an inner surface defining a second channel along a length of the elongate tubular body. A cross-sectional dimension of the first channel may be substantially equal to a cross-sectional dimension of the second channel. A first edge of the first elongate portion may be attached to a first edge of the second elongate portion by a hinge member, and a second edge of the first elongate portion may not be attached to a second edge of the second elongate portion such that the elongate tubular body may move between a first configuration and a second configuration. In the first configuration, the first and second channels may face opposite directions such that the second edge of the first elongate portion may contact the second edge of the second elongate portion. In the second configuration, the first and second channels may face the same direction. The first and second elongate portions may move about the hinge member as the elongate tubular body moves between the first and second configurations. The elongate tubular body may comprise a shape memory material. The hinge member may comprise a flexible, weaker or thinner portion of the shape memory material. The elongate tubular body may comprise a polymeric material. The hinge member may comprise a shape memory material. The medical device may further include at least one retention member disposed along the outer surface of one or both of the first and second elongate portions. A free end of the proximal retention member(s) may face the distal end of the elongate tubular body. A free end of the distal retention member(s) may face the proximal end of the elongate tubular body. The retention member(s) may be disposed along the outer surface of the first or second elongate portions when the elongate tubular body is in the first configuration. The retention member(s) may deflect away from the outer surface of the first or second elongate portion when the elongate tubular body is in the second configuration. The medical device may further include at least one retention member attached to the proximal and/or distal end of the elongate tubular body. The retention member(s) moves from a first configuration to a second configuration as the elongate tubular body moves from the first configuration to the second configuration. The proximal end of the elongate tubular body may include first and second retention members attached thereto, and the distal end of the elongate tubular body may include third and fourth retention members attached thereto. The first retention member may be attached to a proximal end of the first elongate portion. The second retention member may be attached to a proximal end of the second elongate portion, the third retention member may be attached to a distal end of the first elongate portion and the fourth retention member may be attached to a distal end of the second elongate portion. The proximal end of the elongate tubular body may include a first retention member attached thereto, and the distal end of the elongate tubular body may include a second retention member attached thereto. The first retention member may be attached to a proximal end of the first elongate portion, and the second retention member may be attached to a distal end of the first elongate portion. The first retention member may be attached to a proximal end of the first elongate portion, and the second retention member may be attached to a distal end of the second elongate portion. The proximal end of the elongate tubular body may include first and second retention members attached thereto, wherein the first retention member may be attached to a proximal end of the first elongate portion, and the second retention member may be attached to a proximal end of the second elongate portion. The proximal end of the elongate tubular body may include a first retention member attached thereto, wherein the first retention member may be attached to a proximal end of the first elongate portion. The first elongate portion may extend proximally beyond the second elongate portion at a proximal end of the elongate tubular body, and the second elongate portion may extend distally beyond the first elongate portion at the distal end of the elongate tubular body.

In another aspect, the present disclosure relates to a medical device comprising an elongate tubular body which includes a proximal end and a distal end, with a first elongate portion of the elongate tubular body comprising an outer surface and an inner surface defining a first channel along a length of the elongate tubular body, and a second elongate portion of the elongate tubular body comprising an outer surface and an inner surface defining a second channel along a length of the elongate tubular body. A cross-sectional dimension of the first channel may be different than (e.g., greater than, or less than) a cross-sectional dimension of the second channel. A first edge of the first elongate portion may be attached to a first edge of the second elongate portion by a hinge member, and a second edge of the first elongate portion may not be attached to a second edge of the second elongate portion such that the elongate tubular body may move between a first configuration to a second configuration. In the first configuration, the first and second channels may face opposite directions such that the second edge of the first elongate portion may overlap the second edge of the second elongate portion. In the second configuration, the first and second channels may face the same direction. In the first configuration, the first and second channels may face the same direction such that the outer surface of the second elongate portion is received by the first channel of the first elongate portion. In the second configuration, the first and second channels may face opposite directions. The first and second elongate portions may move about the hinge member as the elongate tubular body moves between the first and second configurations. The elongate tubular body may comprise a shape memory material. The hinge member may comprise a flexible, weaker or thinner portion of the shape memory material. The elongate tubular body may comprise a polymeric material. The hinge member comprises a shape memory material. The medical device may further include at least one retention member disposed along the outer surface of one or both of the first and second elongate portions. A free end of the proximal retention member(s) may face the distal end of the elongate tubular body. A free end of the distal retention member(s) may face the proximal end of the elongate tubular body. The retention member(s) may be disposed along the outer surface of the first or second elongate portions when the elongate tubular body is in the first configuration. The retention member(s) may deflect away from the outer surface of the first or second elongate portion when the elongate tubular body is in the second configuration. The medical device may further include at least one retention member attached to the proximal and/or distal end of the elongate tubular body. The retention member(s) may move from a first configuration to a second configuration as the elongate tubular body moves from the first configuration to the second configuration. The proximal end of the elongate tubular body may include first and second retention members attached thereto, and the distal end of the elongate tubular body may include third and fourth retention members attached thereto. The first retention member may be attached to a proximal end of the first elongate portion, the second retention member may be attached to a proximal end of the second elongate portion, the third retention member may be attached to a distal end of the first elongate portion and the fourth retention member may be attached to a distal end of the second elongate portion. The proximal end of the elongate tubular body may include a first retention member attached thereto, and the distal end of the elongate tubular body may include a second retention member attached thereto. The first retention member may be attached to a proximal end of the first elongate portion, and the second retention member may be attached to a distal end of the first elongate portion. The first retention member may be attached to a proximal end of the first elongate portion, and the second retention member may be attached to a distal end of the second elongate portion. The proximal end of the elongate tubular body may include first and second retention members attached thereto, wherein the first retention member may be attached to a proximal end of the first elongate portion, and the second retention member may be attached to a proximal end of the second elongate portion. The proximal end of the elongate tubular body may include first a retention member attached thereto, wherein the first retention member may be attached to a proximal end of the first elongate portion. The first elongate portion may extend proximally beyond the second elongate portion at a proximal end of the elongate tubular body, and the second elongate portion may extend distally beyond the first elongate portion at the distal end of the elongate tubular body.

In another aspect, the present disclosure relates to a method, comprising introducing a medical device, which includes an elongate tubular body within a delivery tube, through a first and second body lumen of a patient, positioning a distal end of the delivery tube within the second body lumen, deploying a first portion of the elongate tubular body within the second body lumen, retracting the delivery tube such that the distal end thereof is disposed within the first body lumen, and deploying a second portion of the elongate tubular body within the first body lumen. The elongate tubular body may move from a first configuration to a second configuration when deployed from the delivery tube.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIGS. 1A-1C provide perspective views of a medical device, according to one embodiment of the present disclosure.

FIGS. 2A-2B provide perspective views of a medical device, according to one embodiment of the present disclosure.

FIGS. 3A-3C provide perspective views of a medical device, according to one embodiment of the present disclosure.

FIGS. 4A-4B provide perspective views of a medical device, according to one embodiment of the present disclosure.

FIGS. 5A-5B provide perspective views of a medical device, according to one embodiment of the present disclosure.

FIG. 6 provides a perspective view of a medical device, according to one embodiment of the present disclosure.

FIGS. 7A-7E provide perspective views of a medical device, according to one embodiment of the present disclosure.

### DETAILED DESCRIPTION

The present disclosure is not limited to the particular embodiments described herein. The terminology used herein is for the purpose of describing particular embodiments only, and is not intended to be limiting beyond the scope of the appended claims. Unless otherwise defined, all technical terms used herein have the same meaning as commonly understood by one of ordinary skill in the art to which the disclosure belongs.

Although embodiments of the present disclosure are described with specific reference to medical devices (e.g., stents, etc.) and methods for drainage of a pancreatic pseudocyst, it should be appreciated that such device and methods may be used in a variety of medical procedures (e.g., enteroenterostomy, gastrojejumostomy, gastroduodenostomy and gastroileostomy, etc.) to establish or maintain a temporary or permanent open flow or access passage between a variety of body organs, lumens, ducts, fistulas, cysts and spaces (e.g., the dermis, stomach, duodenum, jejunum, small intestine, gallbladder, kidneys, pancreas, biliary/pancreatic trees, bladder, ureter, walled-off pancreatic necrosis, abscesses, etc.). Moreover, such medical devices are not limited to solo use, but may facilitate or enhance drainage through one or more previously or simultaneously implanted medical devices, including, for example, an expandable metallic or non-metallic stent.

As used herein, the singular forms “a,” “an,” and “the” are intended to include the plural forms as well, unless the context clearly indicates otherwise. It will be further understood that the terms “comprises” and/or “comprising,” or “includes” and/or “including” when used herein, specify the presence of stated features, regions, steps elements and/or components, but do not preclude the presence or addition of one or more other features, regions, integers, steps, operations, elements, components and/or groups thereof.

As used herein, the term “distal” refers to the end farthest away from the medical professional when introducing a device into a patient, while the term “proximal” refers to the end closest to the medical professional when introducing a device into a patient.

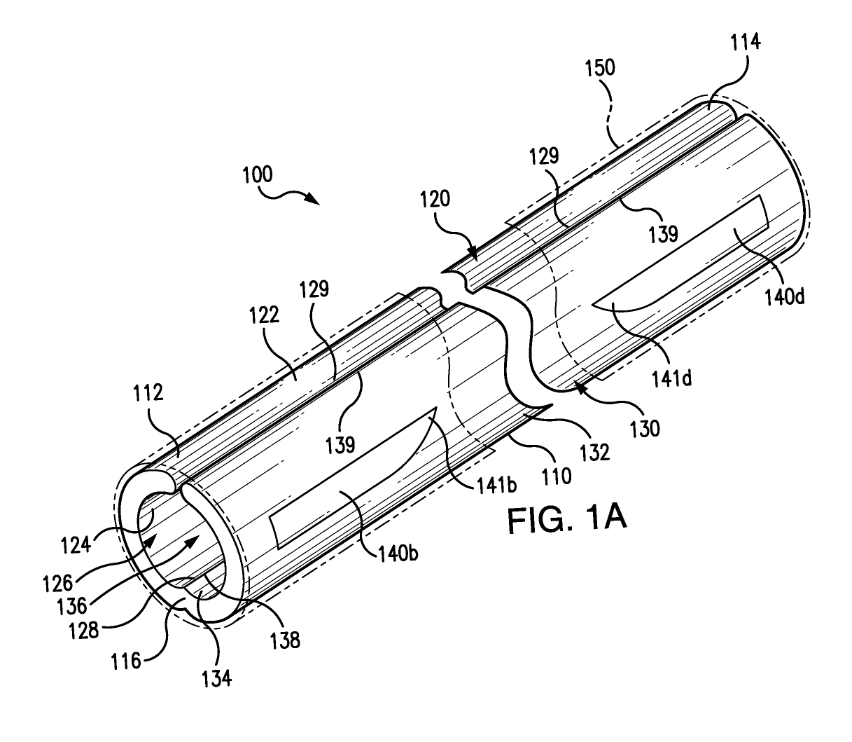

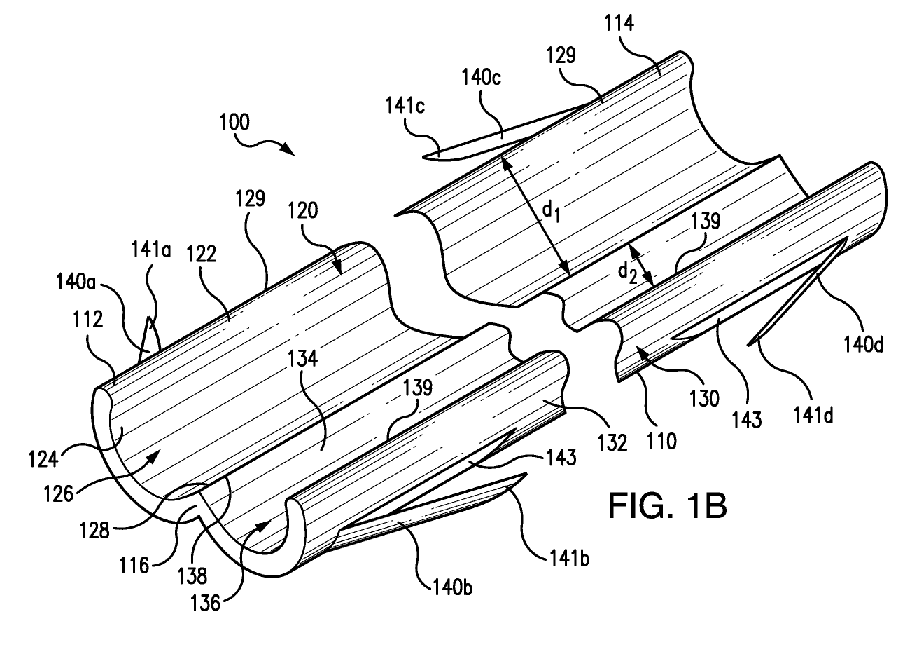

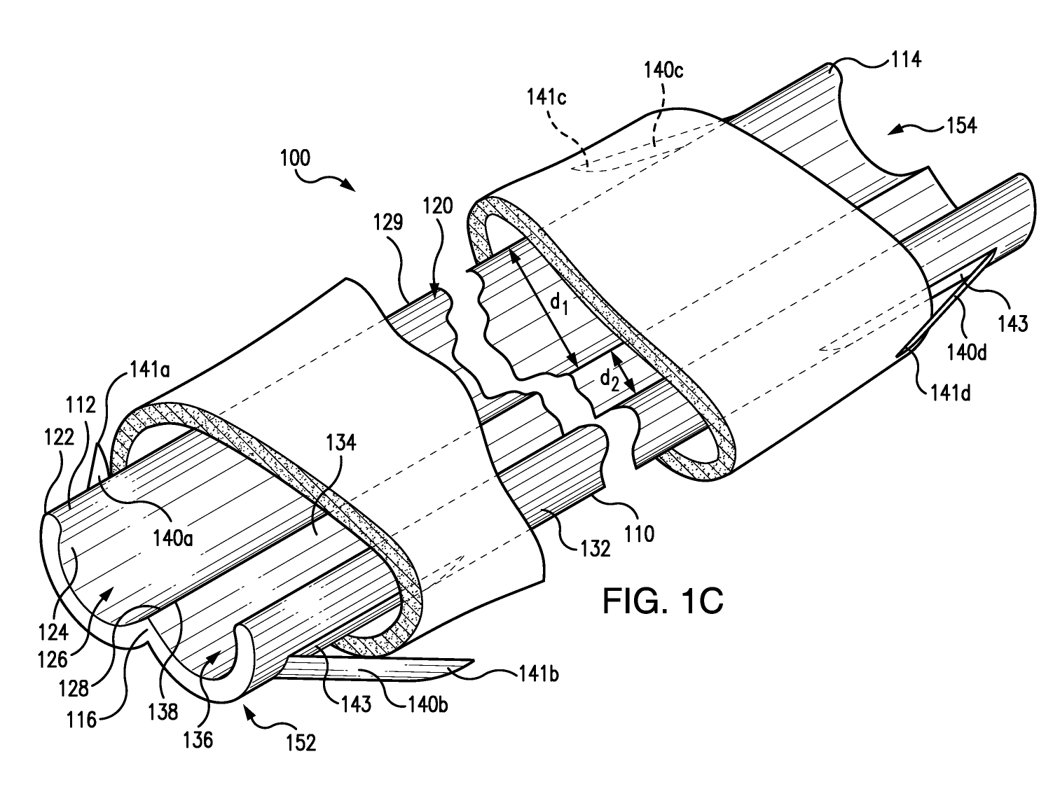

In various embodiments, the present disclosure relates to devices and methods for creating an open flow passage between body lumens and/or within a body vessel. Referring to FIGS. 1A-1C, in one embodiment, a medical device 100 of the present disclosure may include an elongate tubular body 110 comprising a proximal end 112 and a distal end 114. A first elongate portion 120 of the elongate tubular body 110 may include an outer surface 122 and an inner surface 124 defining a first channel 126 (e.g., groove, U-shaped channel, furrow, trough, recess, conduit, duct, etc.) extending along a length, which may be an entire length (e.g., longitudinal axis), of the medical device 100, and a second elongate portion 130 of the elongate tubular body 110 may include an outer surface 132 and an inner surface 134 defining a second channel 136 (e.g., groove, U-shaped channel, furrow, trough, recess, conduit, duct, etc.) extending along a length, which may be an entire length, of the medical device 100. A first edge 128 of the first elongate portion 120 may be attached to a first edge 138 of the second elongate portion 130 by a hinge member 116, and a second edge 129 of the first elongate portion 120 and a second edge 139 of the second elongate portion 130 may be unattached (e.g., free, separated, etc.).

In one embodiment, the hinge member 116 may extend along an entire length of the medical device 100 to join or connect the first and second elongate portions 120, 130. Alternatively, one or more hinge members 116 may join or connect the first and second elongate portions 120, 130 at one or more locations (e.g., middle portion, proximal portion and/or distal portion) along the length of the medical device 100. In other embodiments, attachment elements other than hinge members may be used (e.g., adhesives, pins).

The elongate tubular body 110 may be configured to move between a first (e.g., constrained, closed, delivery, etc.) configuration and a second (e.g., unconstrained, open, deployed, etc.) configuration as the first and second elongate portions 120, 130 move (e.g., pivot, flex, rotate, etc.) relative to each other about the hinge member 116. For example, the medical device 100 may be slidably (e.g., removably) disposed within a delivery tube or sheath 150 that maintains the elongate tubular body 110 in the first configuration (FIG. 1A). When no longer constrained within the delivery tube or sheath, the elongate tubular body 110 may move to the second configuration (FIGS. 1B and 1C). In various embodiments, the medical device may move to a variety of second configurations depending, for example, on the size of the body lumen or vessel in which the medical device is employed. Stated differently, the medical device may contact a portion of the body lumen wall or body vessel wall to prevent (e.g., constrain) the medical device from moving to the W-shaped configuration depicted in FIGS. 1B-1C.

In one embodiment, the first and second elongate portions 120, 130 of the elongate tubular body 110 may be unitarily formed (e.g., extruded) from a shape memory polymeric material, including, linear block copolymers (e.g., polyurethanes, polyurethanes with ionic components, polyurethanes with mesogenic components made by prepolymer methods), block copolymers comprising polyethylene terephthalate (PET) and/or polyethyleneoxide (PEO), ABA triblock copolymer comprising poly(2-methyl-2-oxazoline) and/or polytetrahydrofuran. Other thermoplastic polymers may include, e.g., linear, amorphous polynorbornene (Norsorex®) and/or organic-inorganic hybrid polymers consisting of polynorbornene units partially substituted by polyhedral oligosilsesquioxane (POSS)). The hinge member 116 may comprise a weaker, thinner or otherwise more flexible portion of the shape memory polymer such that the first and second elongate portions 120, 130 preferably flex or pivot about the hinge member 116 as the elongate tubular body 110 moves between the first and second configurations. In addition, or alternatively, the first and second elongate portions 120, 130 of the elongate tubular body 110 may be individually formed from the same or different shape memory polymeric materials and joined or bonded along their respective first edges 128, 138 using a suitable glue, adhesive, solder or resin, as are known in the art, to provide a hinge member or other attachment element therebetween. Alternatively, the individually formed first and second elongate portions 120, 130 may be attached along their respective first edges 128, 138 by a hinge member 116 that is formed from a suitably flexible, bendable or deformable material that is the same or different than the shape memory polymers(s) of the first or second elongate portions, including, but not limited to non-shape memory polymers, metals or alloys.

In various embodiments, the hinge member 116 disclosed herein is not limited to the medical device 100 depicted in FIGS. 1A-1C, but may be included in any of the medical devices 200, 300, 400, 500, 600, 700, discussed below. In addition, or alternatively, the first and/or second elongate portion may be made from non-shape memory polymeric or metallic components, which may be partially or entirely coated with a shape memory polymeric or metallic material such that the elongate tubular body moves to the second configuration. In addition, or alternatively, one or more biasing elements (e.g., springs, etc.) may be incorporated into, along or within the hinge member to bias the device from the first to second configuration.

In one embodiment, a cross-sectional dimension d₁ of the first channel 126 may be substantially equal to a cross-sectional dimension d₂ of the second channel 136. Stated differently, the first and second channels 126, 136 may be substantially symmetrical and a distance between the hinge member 116 and the second edges 129, 139 of the first and second elongate portions 120, 130 is substantially the same (e.g., the first and second edges are separated from the hinge member by approximately 180 degrees relative to a longitudinal axis of the medical device 100) when the elongate tubular body 110 is in the first configuration (FIG. 1A). In the first configuration, the first and second channels 126, 136 may face opposite directions relative to each other (e.g., face inward or towards each other) such that the second edges 129, 139 of the first and second elongate portions 120, 130 may contact, or almost contact, each other to define a substantially enclosed contiguous lumen with a spherical or circular cross section along an entire length of the medical device 100. In the second configuration, the first and second channels 126, 136 may face substantially the same direction such that the second edges 129, 139 of the first and second elongate portions 120, 130 are separated from each other by approximately 180 degrees relative to a longitudinal axis of the medical device 100 to define a W-shaped channel. In various embodiments, the first and second elongate portions 120, 130 may be separated by any number of degrees (e.g., between 0 degrees and 180 degrees) to define a variety of shapes when in the second (e.g., deployed) configuration. For example, the size of the body lumen or body vessel in which the medical device is deployed may prevent the medical device from fully opening into the configuration depicted in FIGS. 1B and 1C.

In one embodiment, one or more retention members may be disposed along the outer surface of the first and/or second elongate portions. For example, first and second retention members 140a, 140b may be disposed along (e.g., attached to or extending from) the respective outer surfaces 122, 132 of the first and second elongate portions 120, 130 distal to the proximal end 112 of the elongate tubular body 110, and third and fourth retention members 140c, 140d may be disposed along the respective outer surfaces 122, 132 of the first and second elongate portions 120, 130 proximal to the distal end 114 of the elongate tubular body 110. When the elongate tubular body is in the first configuration (FIG. 1A), the first retention member (not shown), second retention member 140b, third retention member (not shown) and fourth retention member 140d, may be compressed (e.g., lay flat) against the respective outer surfaces 122, 132 of the first and second elongate portions 120, 130 such that a free end 141a of the first retention member 140a and a free end 141b of the second retention member 140b point or extend towards the distal end 114 of the elongate tubular body 110, and a free end 141c of the third retention member 140c and a free end 141d of the fourth retention member 140d point or extend toward proximal end 112 of the elongate tubular body 110. To maintain a low delivery profile when in the first configuration, the respective outer surfaces 122, 132 of the first and second elongate portions 120, 130 may further include a recessed portion 143 (FIG. 1B) configured to receive each of the retention members 140a-d. When the elongate tubular body is in the second configuration (FIGS. 1B and 1C), the first, second, third and fourth retention members 140a-d may deflect away from the respective outer surfaces 122, 132 of the first and second elongate portions 120, 130 such that respective free ends 141a-d point or extend at an angle relative to a longitudinal axis of the medical device 100. In one embodiment, when the elongate tubular body 110 is deployed between first and second body lumens 152, 154 in the second configuration to establish an open flow passage therebetween, the first and second retention members 140a, 140b may atraumatically contact or engage an inner portion (e.g., inner surface) of the tissue wall of the first body lumen 152, and the third and fourth retention members 140c, 140d may atraumatically engage an inner portion (e.g., inner surface) of the tissue wall of the second body lumen 154, thereby limiting or preventing movement of (e.g., anchoring or securing) the elongate tubular body 110. Although the medical device 100 of FIGS. 1A-1C include a total of four retention members 140a-d, two each at respective ends of the elongate tubular body 110, in various embodiments, any number of retention members may be disposed about the outer surfaces 122, 132, of the first and/or second elongate portions 120, 130 in a variety of patterns, orientations and/or positions relative to a longitudinal axis of the medical device 100.

In various embodiments, the retention members of the present disclosure are not limited to configurations which engage the tissue walls of first and second body lumens, but may also (or alternatively) engage a portion of the tissue wall of a body vessel to limit or prevent migration of the medical device. In addition, when the elongate tubular body 110 is in the second configuration, the retention members 140a-d of the present disclosure may deflect away from the respective outer surfaces 122, 132 of first and second elongate portions 120, 130 at a variety of angles relative to the longitudinal axis of the medical device. For example, the retention members may deflect less than 90 degrees relative to the longitudinal axis of the medical device 100 (e.g., 10 degrees or more, 20 degrees or more, 30 degrees or more, 40 degrees or more, 50 degrees or more, 60 degrees or more, 70 degrees or more, 80 degrees or more), more than 90 degrees relative to the longitudinal axis of the medical device 100 (e.g., 100 degrees or more, 110 degrees or more, 120 degrees or more, 130 degrees or more, 140 degrees or more, 150 degrees or more, 160 degrees or more, 170 degrees or more) or perpendicular to (e.g., 90 degrees) the longitudinal axis of the medical device 100. In addition, the retention members of the present disclosure are not limited to the shape (e.g., retention members 140a-d) depicted in FIGS. 1A-1C, but may include a variety of shapes, including, but not limited to, hooks, barbs, teeth, fingers, and/or projections with rounded or blunt edges or surfaces. In various embodiments, the retention members disclosed herein are not limited to the medical 100 depicted in FIGS. 1A-1C, but may be included in any of the medical devices 200, 300, 400, 500, 600 or 700, disclosed herein.

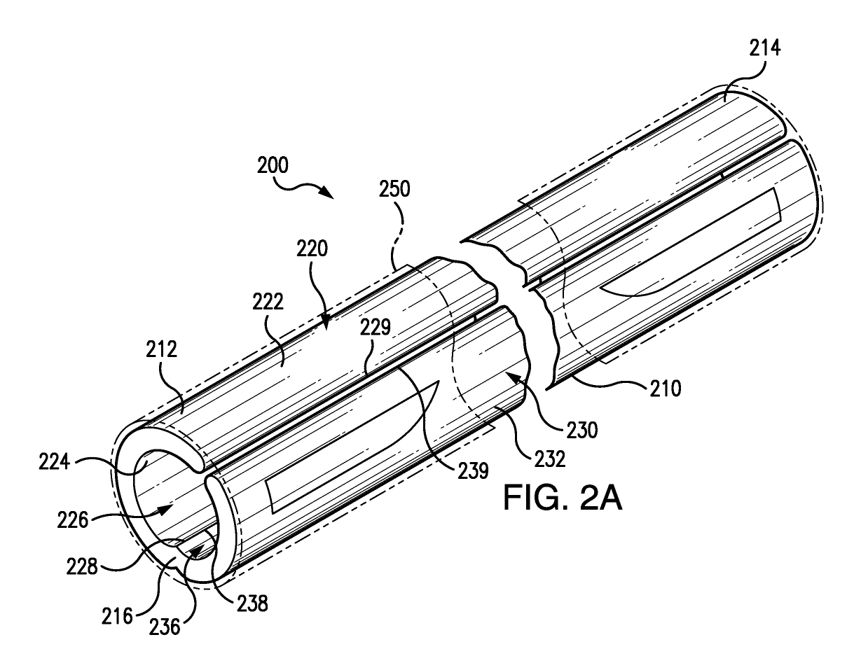

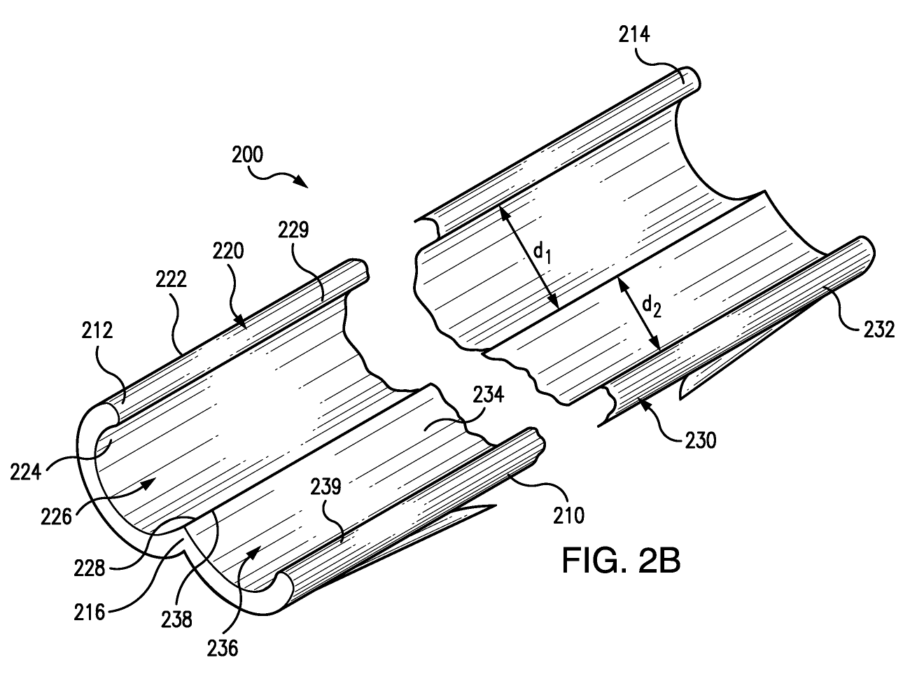

Referring to FIGS. 2A-2B, in one embodiment, a medical device 200 of the present disclosure may include an elongate tubular body 210 comprising a proximal end 212 and a distal end 214. A first elongate portion 220 of the elongate tubular body 210 may include an outer surface 222 and an inner surface 224 defining a first channel 226 (e.g., groove, U-shaped channel, furrow, trough, recess, conduit, duct, etc.) extending along an entire length (e.g., longitudinal axis) of the medical device 200, and a second elongate portion 230 of the elongate tubular body 210 may include an outer surface 232 and an inner surface 234 defining a second channel 236 (e.g., groove, U-shaped channel, furrow, trough, recess, conduit, duct, etc.) extending along an entire length of the medical device 200. A first edge 228 of the first elongate portion 220 may be attached to a first edge 238 of the second elongate portion 230 by a hinge member 216, and a second edge 229 of the first elongate portion 220 and a second edge 239 of the second elongate portion 230 may be unattached (e.g., free, separated, etc.). The elongate tubular body 210 may be configured to move between a first (e.g., constrained, closed, delivery, etc.) configuration and a second (e.g., unconstrained, open, deployed, etc.) configuration as the first and second elongate portions 220, 230 move (e.g., pivot, flex, rotate, etc.) relative to each other about the hinge member 216. For example, the medical device 200 may be slidably (e.g., removably) disposed within a delivery tube or sheath 250 that maintains the elongate tubular body 210 in the first configuration (FIG. 2A). When no longer constrained within the delivery tube or sheath, the elongate tubular body 210 may move to the second configuration (FIG. 2B).

A cross-sectional dimension d₁ of the first channel 226 may be greater than a cross-sectional dimension d₂ of the second channel 236. Stated differently, the first and second channels 226, 236 may be substantially asymmetrical and a distance between the hinge member 216 and the second edge 229 of the first elongate portion 220 is greater than a distance between the hinge member 216 and the second edge 239 of the second elongate portion 230 when the elongate tubular body 310 is in the first configuration (FIG. 2A). In the first configuration, the first and second channels 226, 236 may face opposite directions relative to each other (e.g., face inward or towards each other) such that the second edges 229, 239 of the first and second elongate portions 220, 230 may contact, or almost contact, each other to define a substantially enclosed contiguous lumen with a spherical or circular cross section along a length, which may be an entire length, of the medical device 200. In the second configuration, the first and second channels 226, 236 may face substantially the same direction such that the second edges 229, 239 of the first and second elongate portions 220, 230 are separated from each other by approximately 180 degrees relative to a longitudinal axis of the medical device 100. In various embodiments, the first and second elongate portions 220, 230 may be separated by any number of degrees (e.g., 10 degrees or more, 20 degrees or more, 30 degrees or more, 40 degrees or more, 50 degrees or more, 60 degrees or more, 70 degrees or more, 80 degrees or more, 90 degrees or more, 100 degrees or more, 110 degrees or more, 120 degrees or more, 130 degrees or more, 140 degrees or more, 150 degrees or more, 160 degrees or more, 170 degrees or more, 180 degrees or more, 190 degrees or more, 200 degrees or more) to define a variety of shapes when in the second (e.g., deployed) configuration. For example, the size of the body lumen or body vessel in which the medical device is deployed may prevent the medical device from fully opening into the second configuration depicted in FIG. 2B.

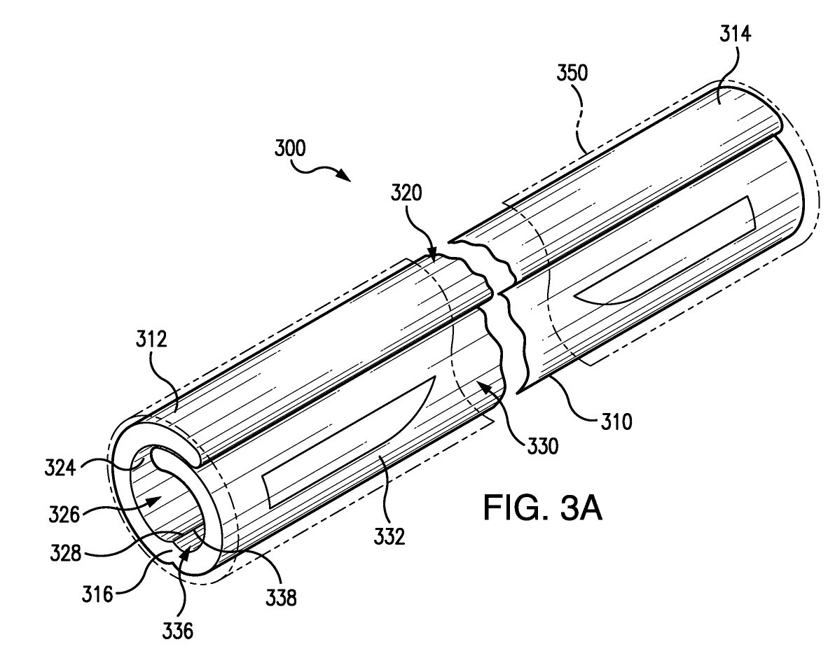

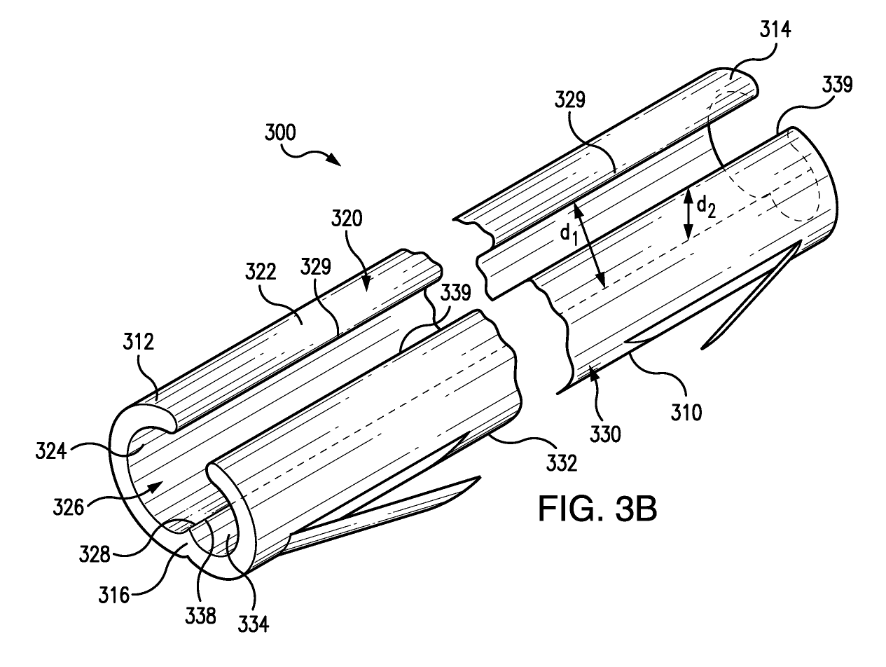

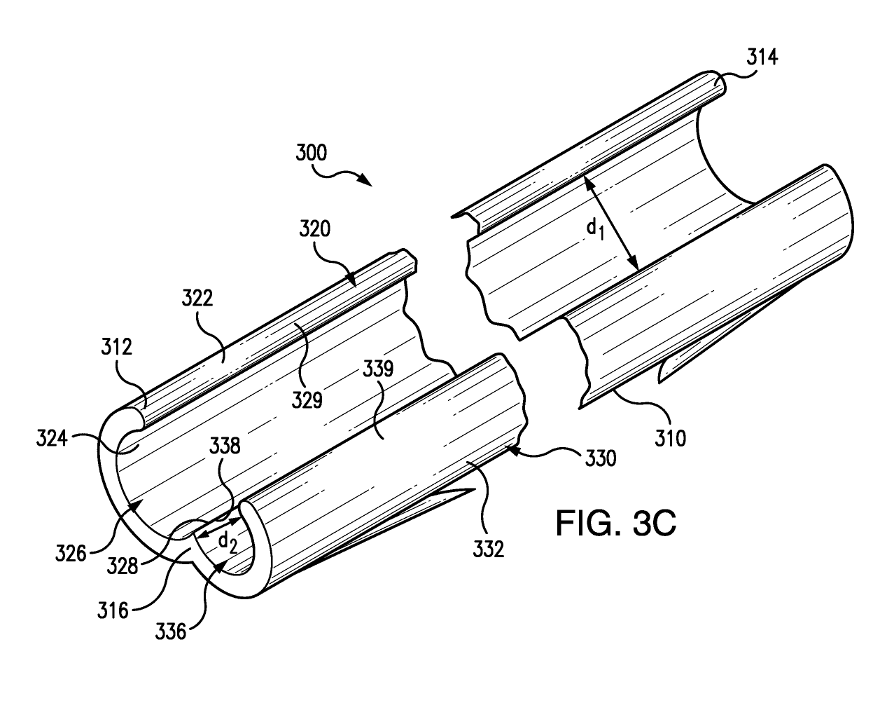

Referring to FIGS. 3A-3C, in one embodiment, a medical device 300 of the present disclosure may include an elongate tubular body 310 comprising a proximal end 312 and a distal end 314. A first elongate portion 320 of the elongate tubular body 310 may include an outer surface 322 and an inner surface 324 defining a first channel 326 (e.g., groove, U-shaped channel, furrow, trough, recess, conduit, duct, etc.) extending along a length (e.g., longitudinal axis) of the medical device 300, and a second elongate portion 330 of the elongate tubular body 310 may include an outer surface 332 and an inner surface 334 defining a second channel 336 (e.g., groove, U-shaped channel, furrow, trough, recess, conduit, duct, etc.) extending along a length of the medical device 300. A first edge 328 of the first elongate portion 320 may be attached to a first edge 338 of the second elongate portion 330 by a hinge member 316, and a second edge 329 of the first elongate portion 320 and a second edge 339 of the second elongate portion 330 may be unattached (e.g., free, separated, etc.). The elongate tubular body 310 may be configured to move between a first (e.g., constrained, closed, delivery, etc.) configuration and a second (e.g., unconstrained, open, deployed) configuration as the first and second elongate portions 320, 330 move (e.g., pivot, flex, rotate, etc.) relative to each other about the hinge member 316. For example, the medical device 300 may be slidably (e.g., removably) disposed within a delivery tube or sheath 350 that maintains the elongate tubular body 310 in the first configuration (FIG. 3A). When no longer constrained within the delivery tube or sheath, the elongate tubular body 310 may move to the second configuration (FIG. 3B).

A cross-sectional dimension d₁ of the first channel 326 may be greater than a cross-sectional dimension d₂ of the second channel 336. Stated differently, the first and second channels 326, 336 may be substantially asymmetrical and a distance between the hinge member 316 and the second edge 329 of the first elongate portion 320 is greater than a distance between the hinge member 316 and the second edge 339 of the second elongate portion 330 when the elongate tubular body 310 is in the first configuration (FIG. 3A). In the first configuration, the first and second channels 326, 336 may face opposite directions relative to each other (e.g., face inward or towards each other) such that the second edge 329 of the first elongate portion 320 overlaps the second edge 339 of the second elongate portion 330 to place a portion of the outer surface 332 of the second elongate portion 330 in contact with, or almost in contact with, a portion of the inner surface 324 of the first elongate portion 320 to define a substantially enclosed contiguous lumen with an asymmetrical (e.g., non-spherical or non-circular) cross section along an entire length of the medical device 300.

In various embodiments, the overlapping portions of the first and second elongate portion 320, 330 may provide a smaller outer diameter (e.g., reduced delivery profile) for placement of the medical device 300 within, or navigation through, small or tortuous body lumens or body vessels. In the second configuration, the first and second channels 326, 336 may face substantially the same direction such that the second edges 329, 339 of the first and second elongate portions 320, 330 are separated from each other by approximately 180 degrees relative to a longitudinal axis of the medical device 300. In various embodiments, the first and second elongate portions 320, 330 may be separated by any number of degrees (e.g., between 0 degrees and 180 degrees) to define a variety of shapes when in the second (e.g., deployed configuration). For example, the size of the body lumen or body vessel in which the medical device is deployed may prevent the medical device from fully opening into the configuration depicted in FIGS. 3B and 3C.

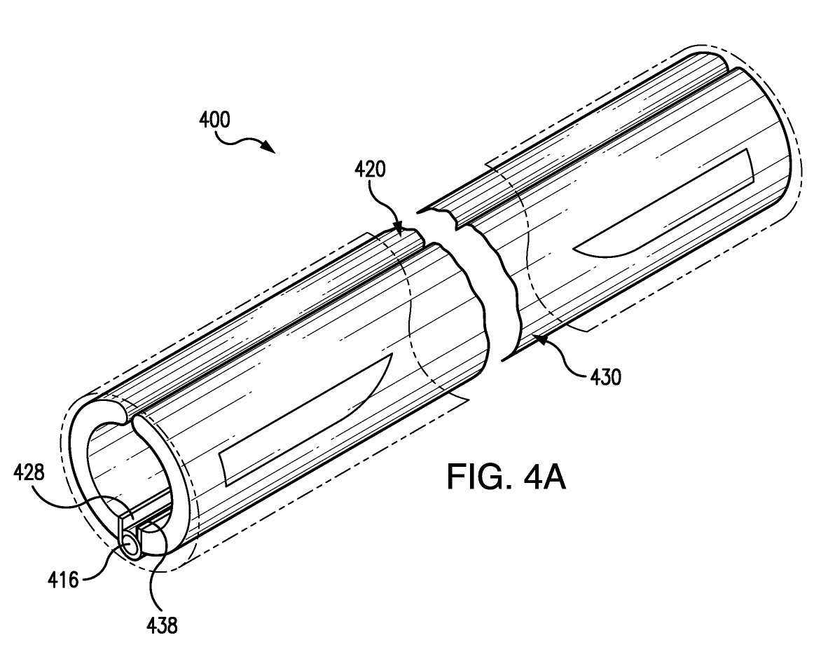

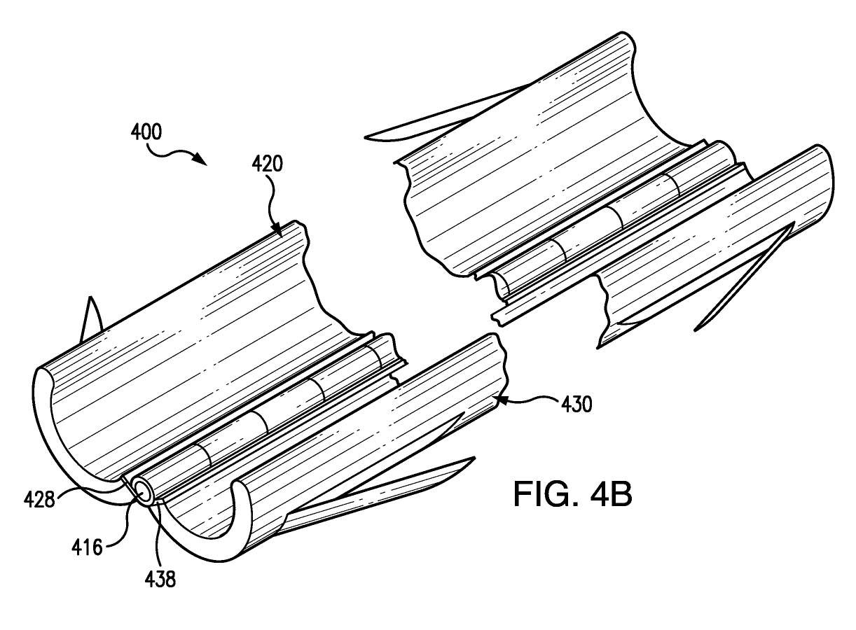

Referring to FIGS. 4A-4B, in one embodiment, a hinge member 416 of the present disclosure may be formed from a shape memory material that extends along all or a portion of a length of the medical device 400 to join or connect the first and second elongate portions 420, 430. For example, the first and second elongate portions 420, 430 of the elongate tubular body 410 may be individually formed from the same or different non-shape memory polymeric materials and joined or bonded along their respective first edges 428, 438 (e.g., using a suitable glue, adhesive, solder or resin, as are known in the art) to a hinge member 416 that is formed from a shape memory polymer, metal or alloy. Alternatively, one or more hinge members 416 may join or connect the first and second elongate portions 420, 430 at one or more locations (e.g., middle portion, proximal portion and/or distal portion) along the length of the medical device 400. The elongate tubular body 410 may be configured to move between a first (e.g., constrained, closed, delivery, etc.) configuration (FIG. 4A) and a second (e.g., unconstrained, open, deployed, etc.) configuration (FIG. 4B) as the first and second elongate portions 420, 430 move (e.g., pivot, flex, rotate, etc.) relative to each other about the hinge member 416. In another embodiment, the elongate tubular body 410 may be formed by co-extruding a non-shape memory polymeric material to form the first second elongate portions 420, 430, with a shape memory polymeric material to form the hinge member 416, such that the first and second elongate portions preferably flex or pivot about the shape memory hinge member 416 as the elongate tubular body 410 moves between the first and second configurations. In various embodiments, the hinge member 416 disclosed herein is not limited to the medical 400 depicted in FIGS. 4A-4B, but may be included in any of the medical devices 100, 200, 300, 500, 600 or 700, disclosed herein.

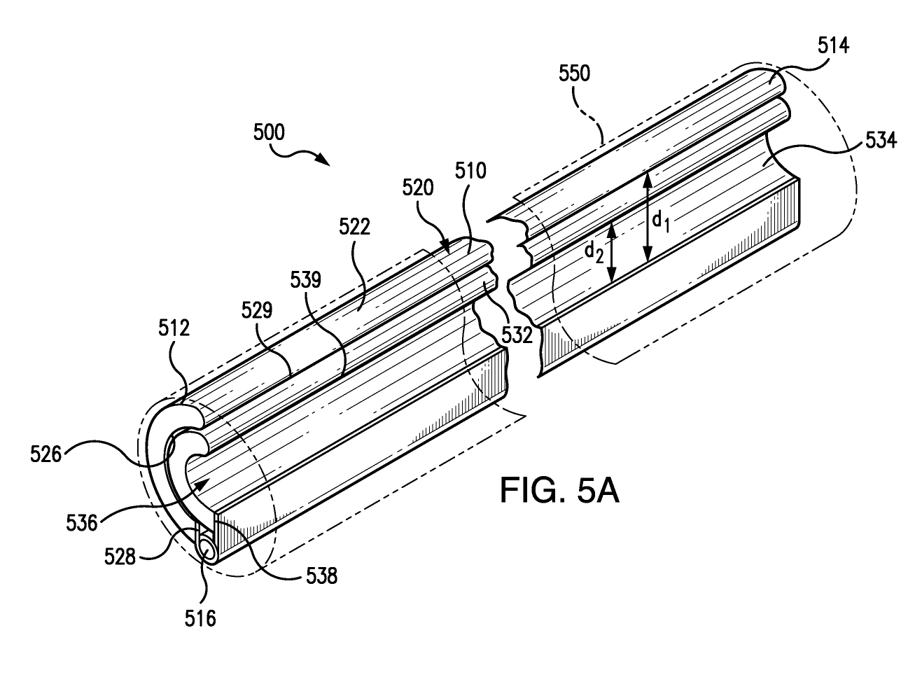

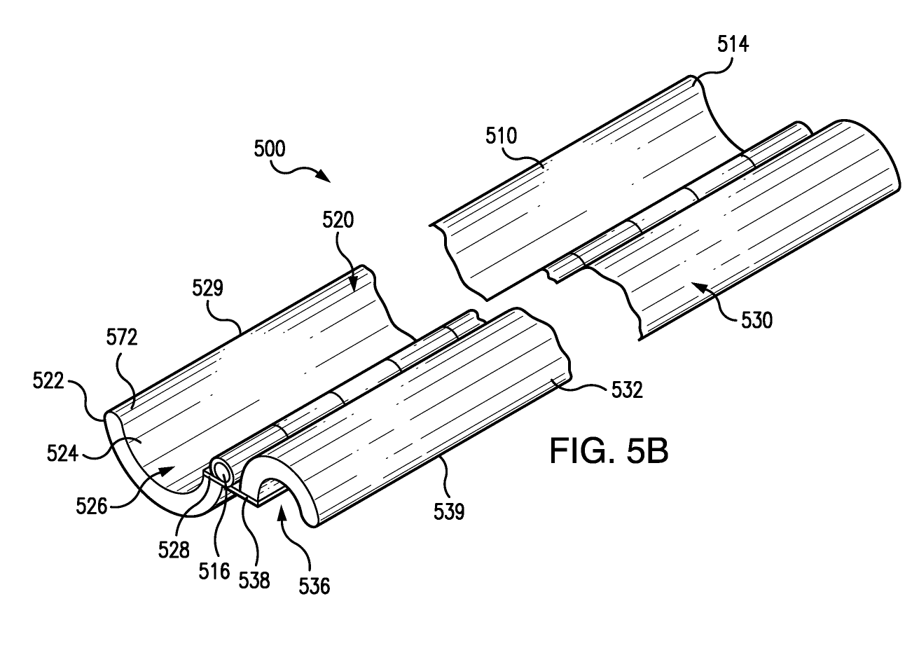

Referring to FIGS. 5A-5B, in one embodiment, a medical device 500 of the present disclosure may include an elongate tubular body 510 comprising a proximal end 512 and a distal end 514. A first elongate portion 520 of the elongate tubular body 510 may include an outer surface 522 and an inner surface 524 defining a first channel 526 (e.g., groove, U-shaped channel, furrow, trough, recess, conduit, duct, etc.) extending along an entire length (e.g., longitudinal axis) of the medical device 500, and a second elongate portion 530 of the elongate tubular body 510 may include an outer surface 532 and an inner surface 534 defining a second channel 536 (e.g., groove, U-shaped channel, furrow, trough, recess, conduit, duct, etc.) extending along an entire length of the medical device 500. A first edge 528 of the first elongate portion 520 may be attached to a first edge 538 of the second elongate portion 530 by a hinge member 516, and a second edge 529 of the first elongate portion 520 and a second edge 539 of the second elongate portion 530 may be unattached (e.g., free, separated, etc.). The elongate tubular body 510 may be configured to move between a first (e.g., constrained, closed, delivery, etc.) configuration (FIG. 5A) and a second (e.g., unconstrained, open, deployed, etc.) configuration (FIG. 5B) as the first and second elongate portions 520, 530 move (e.g., pivot, flex, rotate, etc.) relative to each other about the hinge member 516. For example, the medical device 500 may be slidably (e.g., removably) disposed within a delivery tube or sheath 550 that maintains the elongate tubular body 210 in the first configuration (FIG. 5A). When no longer constrained within the delivery tube or sheath, the elongate tubular body 510 may move to the second configuration (FIG. 5B).

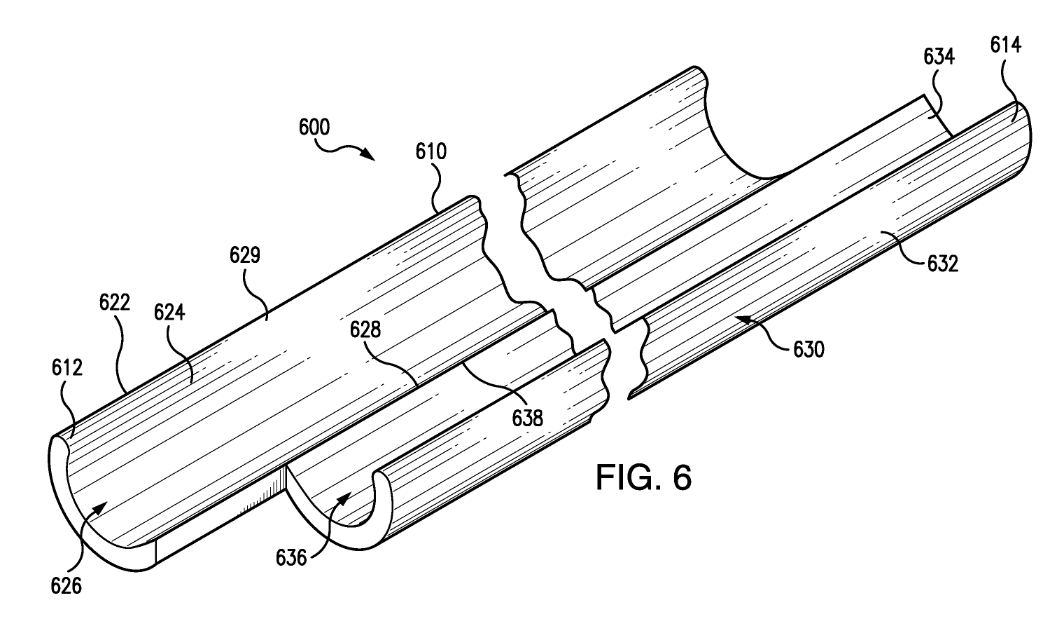

A cross-sectional dimension d₁ of the first channel 526 may be greater than a cross-sectional dimension d₂ of the second channel 536. Stated differently, the first and second channels 526, 536 may be substantially asymmetrical and a distance between the hinge member 516 and the second edge 529 of the first elongate portion 520 is greater than a distance between the hinge member 516 and the second edge 539 of the second elongate portion 530 when the elongate tubular body 510 is in the first configuration (FIG. 5A). The first and second channels 526, 536 may face the same direction relative to each other such that the outer surface 532 of the second elongate portion 530 fits within (e.g., is received by) the first channel 526 of the first elongate portion 520. In various embodiments, in the first configuration (FIG. 6A), the inner surface 524 of the first elongate portion 520 and the outer surface 532 of the second elongate portion 530 may contact, or almost contact, each other to provide a smaller outer diameter (e.g., reduced delivery profile) for placement of the medical device 500 within, or navigation through, small or tortuous body lumens or body vessels. In the second configuration, the first and second channels 526, 536 may face substantially opposite directions (e.g., S-shaped) such that the second edges 529, 539 of the first and second elongate portions 520, 530 are separated from each other by approximately 180 degrees relative to a longitudinal axis of the medical device 500. In various embodiments, the first and second elongate portions 520, 530 may be separated by any number of degrees (e.g., 10 degrees or more, 20 degrees or more, 30 degrees or more, 40 degrees or more, 50 degrees or more, 60 degrees or more, 70 degrees or more, 80 degrees or more, 90 degrees or more, 100 degrees or more, 110 degrees or more, 120 degrees or more, 130 degrees or more, 140 degrees or more, 150 degrees or more, 160 degrees or more, 170 degrees or more, 180 degrees or more, 190 degrees or more, 200 degrees or more) to define a variety of shapes when in the second (e.g., deployed configuration). For example, the size of the body lumen or body vessel in which the medical device is deployed may prevent the medical device from fully opening into the configuration depicted in FIG. 5B.

Referring to FIG. 6, in one embodiment, a medical device 600 of the present disclosure may include an elongate tubular body 610 comprising a proximal end 612 and a distal end 614. A first elongate portion 620 of the elongate tubular body 610 may include an outer surface 622 and an inner surface 624 defining a first channel 626 (e.g., groove, U-shaped channel, furrow, trough, recess, conduit, duct, etc.) extending along a length, which may be an entire length (e.g., longitudinal axis), of the medical device 600, and a second elongate portion 630 of the elongate tubular body 610 may include an outer surface 632 and an inner surface 634 defining a second channel 636 (e.g., groove, U-shaped channel, furrow, trough, recess, conduit, duct, etc.) extending along a length which may be an entire length of the medical device 600. The elongate tubular body 610 may include a staggered configuration in which the first elongate portion 620 extends proximally beyond the second elongate portion 630 at the proximal end 612 of the elongate tubular body 610, and the second elongate portion 630 extends distally beyond the first elongate portion 620 at the distal end 614 of the elongate tubular body 260. A first edge 628 of the first elongate portion 620 may be attached to a first edge 638 of the second elongate portion 630 by a hinge member 616, and a second edge 629 of the first elongate portion 620 and a second edge 639 of the second elongate portion 630 may be unattached (e.g., free, separated, etc.). The elongate tubular body 610 may be configured to move between a first (e.g., constrained, closed, delivery, etc.) configuration (not shown) and a second (e.g., unconstrained, open, deployed, etc.) configuration as the first and second elongate portions 620, 630 move (e.g., pivot, flex, rotate, etc.) relative to each other about the hinge member 616. When the medical device 600 is in the second configuration, an inner portion of the elongate tubular body 610 may include adjacent or side-by-side first and second channels 626, 626, a proximal portion of the elongate tubular body 610 may include a single first channel 626 extending from the first elongate portion 620, and a distal portion of the elongate tubular body 610 may include a single second channel 636 extending from the second elongate portion 630, such that the single first and second channels 626, 636 are offset from each other relative to a longitudinal axis of the medical device 600. This staggered configuration may find beneficial use for deployment of a medical device 600 between body lumens, or within a body vessel, that include a curved or angled architecture, e.g., the various alternative anatomy variations of the extrahepatic biliary tree. In various embodiments, the staggered configuration is not limited to the medical device 600 depicted in FIG. 6, but may be included in any of the medical devices 100, 200, 300, 400, 500 or 700 disclosed herein.

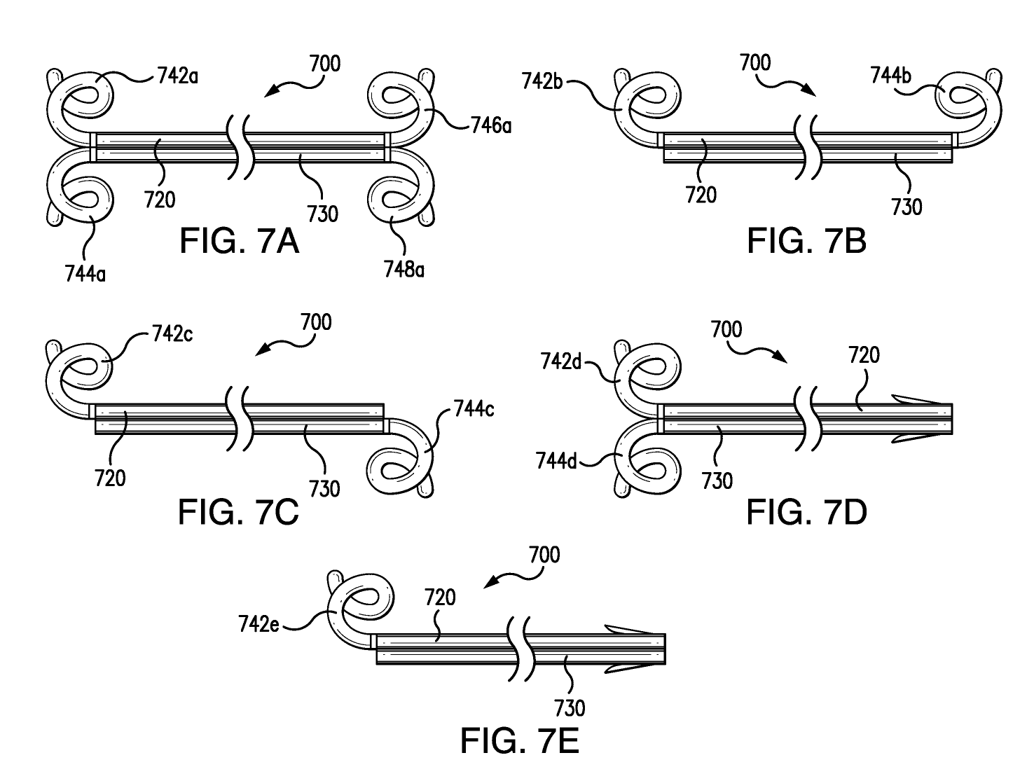

Referring to FIGS. 7A-7B, in one embodiment, any of the medical devices 100, 200, 300, 400, 500, 600 may further include at least one retention member attached to a proximal and/or distal end of the elongate tubular body. By way of non-limiting example, the proximal and/or distal retention members may be configured to move from a first (e.g., linear or straight) configuration to a second (e.g., curved, looped, pigtail, etc.) configuration as the elongate tubular body moves from the first to second configuration (as discussed above). In various embodiments, the retention members may be configured to anchor the respective proximal and/or distal ends of the medical device within the first or second body lumen, thereby minimizing or preventing movement (e.g., migration) of the medical device between body lumens. Although the retention members of FIGS. 7A-7B are solid, in various embodiments, the retention members may define an inner lumen extending along the entire length thereof and include a proximal or distal opening within an end portion of the retention member to place the first and second channels of the elongate tubular body in fluid communication with the retention member(s). In addition, the retention members may further include one or more ports (e.g., openings, holes, etc.) extending through the surface of the retention member to fluidly communicate with the inner lumen.

Referring to FIG. 7A, in one embodiment, a medical device 700 of the present disclosure may include retention members attached to both ends of the first and second elongate portion 720, 730. For example, a first retention member 742a may be attached to a proximal end of the first elongate portion 720, a second retention member 744a may be attached to a proximal end of the second elongate portion 730, a third retention member 746a may be attached to a distal end of the first elongate portion 720 and a fourth retention member 748a may be attached to a distal end of the second elongate portion 730.

Referring to FIG. 7B, in one embodiment, a medical device 700 of the present disclosure may include retention members attached to both ends of the first elongate portion 720, without any retention members attached to the second elongate portion 730. For example, a first retention member 742b may be attached to a proximal end of the first elongate portion 720, and a second retention member 744b may be attached to a distal end of the first elongate portion 720. Alternatively, a first retention member 742b may be attached to a proximal end of the second elongate portion 730, and a second retention member 744b may be attached to a distal end of the second elongate portion 720.

Referring to FIG. 7C, in one embodiment, a medical device 700 of the present disclosure may include retention members attached to opposite ends of the first and second elongate portions 720, 730. For example, a first retention member 742c may be attached to a proximal end of the first elongate portion 720, and a second retention member 744c may be attached to a distal end of the second elongate portion 730. Alternatively, a first retention member 742c may be attached to a distal end of the first elongate portion 730, and a second retention member 744c may be attached to a proximal end of the second elongate portion 730.

Referring to FIG. 7D, in one embodiment, a medical device 700 of the present disclosure may include retention members attached to only one end of the first and second elongate portions 720, 730. For example, a first retention member 742d may be attached to a proximal end of the first elongate portion 720, and a second retention member 744d may be attached to a proximal end of the second elongate portion 730. Alternatively, a first retention member 742d may be attached to a distal end of the first elongate portion 720, and a second retention member 744d may be attached to a distal end of the second elongate portion 730.

Referring to FIG. 7E, in one embodiment, a medical device 700 of the present disclosure may include a retention member attached to only one end of either the first or second elongate portion 720, 730. For example, a first retention member 742e may be attached to proximal end of the first elongate portion 720. Alternatively, a first retention member 742e may be attached to proximal end of the second elongate portion 730. Alternatively, a first retention member 742e may be attached to distal end of the first elongate portion 720. Alternatively, a first retention member 742e may be attached to the distal end of the second elongate portion 720.

In various embodiments, the present disclosure relates to methods for creating an open flow passage between two or more structures (e.g., a first body lumen and a second body lumen). In use and by way of example, any of the medical devices disclosed herein may be used to establish an open flow passage between body lumens and/or within a body vessel by introducing a medical device comprising an elongate tubular body in a first configuration, e.g., constrained within a delivery tube or sheath, through a first and second body lumen of a patient such that a distal end of the delivery tube is positioned within the second body lumen (e.g., cyst, bladder, etc.). A first portion of the elongate tubular body may then be deployed within the second body lumen such that one or more retention members on a distal end or distal portion of the elongate tubular body move from a first to second configuration within the second body lumen. The delivery tube may then be retracted such that the distal end thereof is disposed within the first body lumen (e.g., stomach, kidney, etc.). A second portion of the elongate tubular body may then be deployed within the first body lumen such that one or more retention members on a proximal end or proximal portion of the elongate tubular body move from a first to second configuration within the first body lumen.

As exemplary methods, a pseudocyst drainage procedure may involve placing a medical device of the present disclosure between a pancreatic pseudocyst, walled-off pancreatic necrosis (WOPN) or pancreatic fluid collection (e.g., second body lumen) and the stomach or duodenum (e.g., first body lumen). A kidney drainage procedure may involve placing a medical device of the present disclosure between the kidney (e.g., second body lumen) and the bladder (e.g., first body lumen). A gallbladder drainage procedure may involve placing a medical device of the present disclosure between the gallbladder (e.g., second body lumen) and the stomach (e.g., first body lumen). A cholecystoduodenostomy procedure may involve placing a medical device of the present disclosure between the gallbladder (e.g., second body lumen) and the duodenum (e.g., first body lumen). Alternatively, a percutaneous cholecystoduodenostomy procedure may involve placing a medical device of the present disclosure between the gallbladder (e.g., second body lumen) and the jejunum (e.g., first body lumen). An enteroenterostomy procedure may involve placing a medical device of the present disclosure between a first portion of the intestine (e.g., second body lumen) and a second portion of the intestine (e.g., first body lumen). A choledochoduodenostomy procedure may involve placing a medical device of the present disclosure between the common biliary duct (e.g., second body lumen) and duodenum (e.g., first body lumen). A gastroileostomy procedure may involve placing a medical device of the present disclosure between the stomach (e.g., second body lumen) and ileum (e.g., first body lumen). A gastroduodenstomy procedure may involve placing a medical device of the present disclosure between the stomach (e.g., second body lumen) and duodenum (e.g., first body lumen). A gastrojejumostomy procedure may involve placing a medical device of the present disclosure between the stomach (e.g., second body lumen) and jejunum (e.g., first body lumen).

The medical devices depicted in FIGS. 1A-7E are provided by way of non-limiting example, and may include a variety of different shapes, configurations, orientations, dimensions and/or materials as required to provide an open flow or access pathway between first and second body lumens or within a body vessel. Variations on these devices and other devices, and associated components and features which may be suitable for the systems and methods of the present disclosure, can be found in U.S. application Ser. No. 14/315,910, the entire disclosure of which are herein incorporated by reference in its entirety.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A medical device, comprising:
an elongate tubular body comprising a proximal end and a distal end;
a first elongate portion of the elongate tubular body comprising an outer surface and an inner surface defining a first channel along a length of the elongate tubular body;
a second elongate portion of the elongate tubular body comprising an outer surface and an inner surface defining a second channel along a length of the elongate tubular body;
wherein a cross-sectional dimension of the first channel is substantially equal to a cross-sectional dimension of the second channel;
wherein a first edge of the first elongate portion is attached to a first edge of the second elongate portion by a hinge member;
wherein a second edge of the first elongate portion is not attached to a second edge of the second elongate portion; and
wherein the elongate tubular body is configured to move between a first configuration and a second configuration.

2. The medical device of claim 1, wherein in the first configuration, the first and second channels face opposite directions such that the second edge of the first elongate portion contacts the second edge of the second elongate portion.

3. The medical device of claim 1, wherein in the second configuration, the first and second channels face the same direction.

4. The medical device of claim 1, wherein the first and second elongate portions move about the hinge member as the elongate tubular body moves between the first and second configurations.

5. The medical device of claim 1, wherein the elongate tubular body comprises a shape memory material.

6. The medical device of claim 5, wherein the hinge member comprises a flexible, weaker or thinner portion of the shape memory material.

7. The medical device of claim 1, wherein the elongate tubular body comprises a polymeric material.

8. The medical device of claim 7, wherein the hinge member comprises a shape memory material.

9. A medical device, comprising:
an elongate tubular body comprising a proximal end and a distal end;
a first elongate portion of the elongate tubular body comprising an outer surface and an inner surface defining a first channel along a length of the elongate tubular body;
a second elongate portion of the elongate tubular body comprising an outer surface and an inner surface defining a second channel along a length of the elongate tubular body;
wherein a cross-sectional dimension of the first channel is different than a cross-sectional dimension of the second channel;
wherein a first edge of the first elongate portion is attached to a first edge of the second elongate portion by a hinge member;
wherein a second edge of the first elongate portion is not attached to a second edge of the second elongate portion; and
wherein the elongate tubular body is configured to move between a first configuration to a second configuration.

10. The medical device of claim 9, wherein in the first configuration, the first and second channels face opposite directions such that the second edge of the first elongate portion overlaps the second edge of the second elongate portion.

11. The medical device of claim 9, wherein in the second configuration, the first and second channels face the same direction.

12. The medical device of claim 9, wherein in the first configuration, the first and second channels face the same direction such that the outer surface of the second elongate portion is received by the first channel of the first elongate portion.

13. The medical device of claim 9, wherein in the second configuration, the first and second channels face opposite directions.

14. The medical device of claim 9, wherein the first and second elongate portions move about the hinge member as the elongate tubular body moves between the first and second configurations.

15. The medical device of claim 9, wherein the elongate tubular body comprises a shape memory material.

16. The medical device of claim 15, wherein the hinge member comprises a flexible, weaker or thinner portion of the shape memory material.

17. The medical device of claim 9, wherein the elongate tubular body comprises a polymeric material.

18. The medical device of claim 17, wherein the hinge member comprises a shape memory material.

19. A method, comprising:
introducing a medical device through a first and second body lumen of a patient, the medical device comprising an elongate tubular body within a delivery tube;
positioning a distal end of the delivery tube within the second body lumen;
deploying a first portion of the elongate tubular body within the second body lumen;
retracting the delivery tube such that the distal end thereof is disposed within the first body lumen;
deploying a second portion of the elongate tubular body within the first body lumen.

20. The method of claim 19, wherein the elongate tubular body moves from a first configuration to a second configuration when deployed from the delivery tube.
